(19) United States
(12) Patent Application Publication
(10) Pub. No.: US 2008/0226696 A1
(43) Pub. Date: Sep. 18, 2008

(54) Methods of improving learning abilities of aged companion animals

(76) Inventors: Ben Treadwell, Addison, ME (US); Nathan Hamilton, Incline Village, NV (US)

Correspondence Address: MORRISON & FOERSTER LLP, 755 PAGE MILL RD, PALO ALTO, CA, 94304-1018, US

(21) Appl. No.: 11/788,713

(22) Filed: Apr. 20, 2007

Related U.S. Application Data
(60) Provisional application No. 60/918,487, filed on Mar. 15, 2007.

(51) Int. Cl. A61K 31/205; A23K 1/16; A61K 31/385; A61K 31/4188; A61P 25/00

(52) U.S. Cl. 424/442; 514/387; 514/440; 514/561

(57) ABSTRACT

The present invention provides methods of improving learning ability of aged companion animals (such as aged dogs) by administering an effective amount of a carnitine and an effective amount of a lipoic acid. Also provided are kits and animal treats for improving learning ability of aged companion animals comprising effective amounts of carnitine and lipoic acid.

### CROSS-REFERENCE TO RELATED APPLICATIONS

This application claims the priority benefit under 35 U.S.C. §119(e) of U.S. Provisional Patent Application No. 60/918,487, filed Mar. 15, 2007, the content of which is herein incorporated by reference in its entirety.

### TECHNICAL FIELD

This application is in the field of companion animal care, more specifically in the field of improving learning ability of aged companion animals by administration of effective amounts of carnitine and lipoic acid.

### BACKGROUND

Companion animals such as dogs and cats show marked age-dependent decline in learning and memory. The decline in learning and memory are manifested in behavioral problems such as lack of perception and awareness, decreased exploratory activities, sleep disturbance, housetraining deficits, restlessness, disorientation, attention difficulties, decreased motivation, decreased or altered interaction with humans and other companion animals, and increased anxiety. Decline in learning and memory impairs the quality of life of the companion animal, and also impacts the bonds between the companion animal and the animal owner.

Age-related decline in learning and memory creates even bigger problems for companion animals that are specially trained to carry out a uniquely important function, such as assisting physically disabled individuals and performing a variety of military functions. As the companion animals age and become less attentive, new companion animals will have to be trained to replace the old ones.

Use of dietary intervention to improve learning abilities of dogs have previously been reported, for example, in Milgram et al., (2002), Neuroscience and Behavioral Reviews, 26:679-95 and Milgram et al. (2005), Neurobiology of Aging, 26:77-90. Briefly, dog food enriched with a cocktail of antioxidants, including fruit extracts, vegetable extracts, vitamin C, vitamin E, as well as low levels of L-carnitine and lipoic acid, was fed to aged dogs. Landmark tests were used to evaluate the effects of the antioxidant cocktail. Dogs fed with the antioxidant fortified food, when compared with control dogs, showed improved learning ability within one or more month(s) from the start of treatment. Because a large number of ingredients and antioxidants were fed to the dogs, it was unclear which, if any, specific component is particularly important. It has been postulated that the observed effects on learning depend on a broad spectrum of ingredients in the antioxidant cocktail. Milgram et al. (2005), Neurobiology of Aging, 26:77-90. U.S. Pat. No. 6,914,071 discloses inhibiting onset of deterioration of the mental capacity of companion animals in their aged years by maintaining the companion animals on an antioxidant-fortified diet during their adult years (i.e., 6 years old or younger).

Use of carnitines has been described in U.S. Pat. No. 5,362,753 (Method of increasing the hatchability of eggs by feeding hens carnitine); U.S. Pat. No. 4,687,782 (Nutritional composition for enhancing skeletal muscle adaptation to exercise training); U.S. Pat. No. 5,030,458 (Method for preventing diet-induced carnitine deficiency in domesticated dogs and cats); U.S. Pat. No. 5,030,657 (L-carnitine supplemented catfish diet); U.S. Pat. No. 4,343,816 (Pharmaceutical composition comprising an acyl-carnitine for treating peripheral vascular diseases); U.S. Pat. No. 5,560,928 (Nutritional and/or dietary composition and method of using the same); U.S. Pat. No. 5,504,072 (Enteral nutritional composition having balanced amino acid profile); U.S. Pat. No. 5,391,550 (Compositions of matter and methods for increasing intracellular ATP levels and physical performance levels and for increasing the rate of wound repair); and U.S. Pat. No. 5,240,961 (Method of treating reduced insulin-like growth factor and bone loss associated with aging).

Use of lipoic acid has been described in U.S. Pat. No. 5,607,980 (Topical compositions having improved skin feel); U.S. Pat. No. 5,472,698 (Composition for enhancing lipid production in skin); U.S. Pat. No. 5,292,538 (Improved sustained energy and anabolic composition and method of making); U.S. Pat. No. 5,536,645 (Nutritive medium for the culture of microorganisms); and U.S. Pat. No. 5,326,699 (Serum-free medium for culturing animal cells).

Compositions comprising carnitine and lipoic acid have been previously disclosed in U.S. Pat. Nos. 5,916,912 and 6,335,361, as well as U.S. Pat. App. Pub. No. 20030060503.

The disclosures of all publications, patents, patent applications and published patent applications referred to herein are hereby incorporated herein by reference in their entirety.

### BRIEF SUMMARY OF THE INVENTION

The present invention in one aspect provides methods of improving learning ability of an aged companion animal (such as an aged dog), comprising administering to the aged companion animal an effective amount of a carnitine (such as an alkanoyl-L-carnitine, for example acetyl-L-carnitine or ALCAR) and an effective amount of a lipoic acid, wherein the amount of carnitine is at least about 8 mg/kg body weight/day (such as about 8-275 mg/kg body weight/day) and wherein the amount of lipoic acid is at least about 4 mg/kg body weight/day (such as about 4-110 mg/kg body weight/day). In some embodiments, the amount of carnitine is about 8-40 mg/kg body weight/day and the amount of lipoic acid is about 4-20 mg/kg body weight/day. In some embodiments, the amount of carnitine is at least about 20 mg/kg body weight/day, and the amount of lipoic acid is at least about 10 mg/kg body weight/day. In some embodiments, the improvement of learning ability of the aged companion animal can be observed within less than about one month after the initiation of the administration, including for example less than about any of three weeks, two weeks, one week, 6 days, 5 days, 4 days, 3 days, 2 days, or one day after the initiation of the administration. In some embodiments, the improvement of learning ability of the aged companion animal can be observed within any of about 2, 3, 4, 5, 8, 10, 12, 18, or 24 hours after the initiation of the administration. In some embodiments, the carnitine and the lipoic acid are administered orally.

In some embodiments, there is provided a method of enhancing learning ability of an aged companion animal, comprising administering to the aged companion animal an effective amount of a carnitine (such as alkanoyl-L-carnitine, for example ALCAR) and an effective amount of a lipoic acid, wherein the amount of carnitine (such as alkanoyl-L-carnitine, for example ALCAR) is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day). In some embodiments, there is provided a method of reversing decline of learning ability of an aged companion animal, comprising administering to the aged companion animal an effective amount of a carnitine (such as alkanoyl-L-carnitine, for example ALCAR) and an effective amount of a lipoic acid, wherein the amount of carnitine (such as alkanoyl-L-carnitine, for example ALCAR) is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day).

In some embodiments, there is provided a method of preventing or delaying further decline of learning ability of an aged companion animal, comprising administering to the aged companion animal an effective amount of a carnitine (such as alkanoyl-L-carnitine, for example ALCAR) and an effective amount of a lipoic acid, wherein the amount of carnitine (such as alkanoyl-L-carnitine, for example ALCAR) is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day). In some embodiments, there is provided a method of retarding decline of learning ability of an aged companion animal, comprising administering to the aged companion animal an effective amount of a carnitine (such as alkanoyl-L-carnitine, for example ALCAR) and an effective amount of a lipoic acid, wherein the amount of carnitine (such as alkanoyl-L-carnitine, for example ALCAR) is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day).

In some embodiments, there is provided a method of reducing one or more signs associated with decline of learning ability of an aged companion animal, comprising administering to the aged companion animal an effective amount of a carnitine (such as alkanoyl-L-carnitine, for example ALCAR) and an effective amount of a lipoic acid, wherein the amount of carnitine (such as alkanoyl-L-carnitine, for example ALCAR) is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day).

In some embodiments, the amount of carnitine is in the range of about 8-40 mg/kg body weight/day, including for example about 10-35 mg/kg body weight/day, about any of 15-30 mg/kg body weight/day, about 20-28 mg/kg body weight/day, or about 27.5 mg/kg body weight/day. In some embodiments, the carnitine is an alkanoyl-L-carnitine. In some embodiments, the carnitine is ALCAR. In some embodiments, the amount of lipoic acid is in the range of about 4-20 mg/kg body weight/day, including for example about 8-15 mg/kg body weight/day, about 10-15 mg/kg body weight/day, or about 11 mg/kg body weight/day. In some embodiments, the lipoic acid is enriched with R-lipoic acid. In some embodiments, the lipoic acid is a mixture of R-lipoic acid and S-lipoic acid having an R-enantiomer excess of at least about 40%. In some embodiments, the lipoic acid is R-lipoic acid.

In some embodiments, the carnitine and the lipoic acid are administered sequentially. In some embodiments, the carnitine and the lipoic acid are administered simultaneously.

In some embodiments, the carnitine and the lipoic acid are administered as a single composition. For example, in some embodiments, there are provided methods of improving learning ability of an aged companion animal, comprising administering to the aged companion animal a composition comprising an effective amount of a carnitine and an effective amount of a lipoic acid, wherein the amount of carnitine is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day). In some embodiments, there is provided a method of enhancing learning ability of an aged companion animal, comprising administering to the aged companion animal a composition comprising an effective amount of a carnitine and an effective amount of a lipoic acid, wherein the amount of carnitine is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day). In some embodiments, there is provided a method of reversing decline of learning ability of an aged companion animal, comprising administering to the aged companion animal a composition comprising an effective amount of a carnitine and an effective amount of a lipoic acid, wherein the amount of carnitine is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day).

In some embodiments, the composition comprises at least about 2% (w/w) (including for example at least about any of 5%, 10%, 20%, 30%, 40%, 50%, 60%, or 70%) carnitine and at least about 1% (w/w) (including for example at least about any of 2%, 3%, 5%, 10%, 20%, 25%, or 30%) lipoic acid. In some embodiments, the weight ratio of the carnitine and the lipoic acid in the composition is about any of 5:1, 4:1, 3:1, 2:1, 1:1, 1:2, 1:3, 1:4, or 1:5. In some embodiments, the composition comprises a synergistic ratio of carnitine and lipoic acid. In some embodiments, the composition further comprises biotin (for example at least about any of 0.001%, 0.01%, 0.02%, 0.05%, 0.1%, 0.5%, 1%, 2%, or 5% (w/w) of biotin).

In some embodiments, the composition comprises less than about any of 5%, 3%, 2%, 1%, 0.5%, or 0.1% antioxidant other than carnitine and lipoic acid. In some embodiments, the composition further comprises any one or more of the following: meat by-product, poultry by-product, meat meal, bone meal, chicken or poultry liver digest, chicken or poultry viscera digest, brewer rice, and brewer dry yeast. In some embodiments, the composition further comprises any one or more of the following: chicken meal, rice flour, wheat flour, corn syrup, water, glycerin, molasses, tapioca starch, gelatin, natural flavor, chicken fat (for example chicken fat preserved with mixed tocopherols and citric acid), salt, phosphoric acid, L-ascorbyl-2-polyphosphate, potassium sorbate, mono and diglycerides, vitamin E supplement, minerals (such as zinc proteinate, iron, proteinate, copper proteinate, manganese proteinate, cobalt proteinate), calcium propionate, garlic powder, and biotin supplement.

In some embodiments, the carnitine and the lipoic acid (in the same or different composition(s)) are administered once a day. In some embodiments, the carnitine and the lipoic acid (in the same or different composition(s)) are administered at least two times daily, including for example, at least about any of 3, 4, 5, 6, 7, or 8 times daily. In some embodiments, the carnitine and the lipoic acid (in the same or different composition(s)) are administered at least about any of 2, 3, 4, 5, 6, 7, 8, 9, or 10 hours after a meal (such as the main meal(s)) of the companion animal. In some embodiments, the carnitine and the lipoic acid (in the same or different composition(s)) are administered at least about any of 0.2, 0.5, 1, 2, 3, 4, 5, or 6 hours prior to the administration of a meal (such as the main meal(s)) of the companion animal.

In some embodiments, the companion animal is a canine or a feline, such as a dog or a cat. In some embodiments, the companion animal is at least about any of 7, 8, 9, 10, 15, 20, 25, or 30 years old. In some embodiments, the companion animal is a dog of small breed that is over about 8 years of age. In some embodiments, the companion animal is a dog of medium breed that is over about 7 years of age. In some embodiments, the companion animal is a dog of large breed that is over 6 years of age. In some embodiments, the companion animal is a dog of giant breed that is over 5 years of age.

In some embodiments, the invention provides a method of improving learning ability of an aged companion animal (such as an aged dog), comprising administering to the aged companion animal (such as aged dog) an effective amount of a carnitine (such as alkanoyl-L-carnitine, for example ALCAR) and an effective amount of a lipoic acid, wherein the amount of carnitine (such as alkanoyl-L-carnitine, for example ALCAR) is about 8-40 mg/kg body weight/day and wherein the amount of lipoic acid is about 4-20 mg/kg body weight/day, wherein the carnitine (such as alkanoyl-L-carnitine, for example ALCAR) and lipoic acid are administered at least two times daily. In some embodiments, the carnitine (such as alkanoyl-L-carnitine, for example ALCAR) and the lipoic acid are administered at least about 2 hours (including for example at least about any of 3, 4, 5, 6, 7, 8, 9, or 10 hours) after a meal (such as the main meal(s)) of the companion animal. In some embodiments, the carnitine and the lipoic acid (in the same or different composition(s) are administered at least about any of 0.2, 0.5, 1, 2, 3, 4, 5, or 6 hours prior to the administration of a meal (such as the main meal(s)) of the companion animal.

In some embodiments, the invention provides a method of improving learning ability of an aged companion animal (such as an aged dog), comprising administering to the aged companion animal (such as an aged dog) a composition comprising an effective amount of a carnitine (such as alkanoyl-L-carnitine, for example ALCAR) and an effective amount of a lipoic acid, wherein the amount of carnitine (such as alkanoyl-L-carnitine, for example ALCAR) is about 8-40 mg/kg body weight/day and wherein the amount of lipoic acid is about 4-20 mg/kg body weight/day, wherein the carnitine (such as alkanoyl-L-carnitine, for example ALCAR) and lipoic acid are administered at least two times daily. In some embodiments, the carnitine (such as alkanoyl-L-carnitine, for example ALCAR) and the lipoic acid are administered at least about 2 hours (including for example at least about any of 3, 4, 5, 6, 7, 8, 9, or 10 hours) after a meal (such as the main meal(s)) of the companion animal. In some embodiments, the carnitine (such as alkanoyl-L-carnitine, for example ALCAR) and the lipoic acid are administered at least about any of 0.2, 0.5, 1, 2, 3, 4, 5, or 6 hours prior to the administration of a meal (such as the main meal(s)) of the companion animal.

In some embodiments, the invention provides a method of improving learning ability of an aged dog, comprising administering to the aged dog an effective amount of ALCAR and an effective amount of a lipoic acid, wherein the ALCAR is about 8-40 mg/kg body weight/day and wherein the amount of lipoic acid is about 4-20 mg/kg body weight/day, wherein the ALCAR and lipoic acid are administered at least two times daily, and wherein the ALCAR and the lipoic acid are administered at least about 2 hours (including for example at least about any of 3, 4, 5, 6, 7, 8, 9, or 10 hours) after a meal (such as the main meal(s)) of the companion animal. In some embodiments, the invention provides a method of improving learning ability of an aged dog, comprising administering to the aged dog an effective amount of ALCAR and an effective amount of a lipoic acid, wherein the ALCAR is about 8-40 mg/kg body weight/day and wherein the amount of lipoic acid is about 4-20 mg/kg body weight/day, wherein the ALCAR and lipoic acid are administered at least two times daily, and wherein the ALCAR and the lipoic acid are administered at least about any of 0.2, 0.5, 1, 2, 3, 4, 5, or 6 hours prior to the administration of a meal (such as the main meal(s)) of the companion animal.

The methods described herein are generally useful for any one or more of the following: increasing vitality of an aged companion animal; increasing perception and awareness of an aged companion animal; increasing exploratory activity of an aged animal; improving quality of life of an aged companion animals and/or a companion animal owner; improving companionship of an aged companion animal; increasing ability of an aged companion animal to respond to tasks, other animals, and people; treating dementia of an aged companion animal; treating age-related deterioration of mental function of an aged companion animal; and preventing, retarding, or delaying development of age-related neuropathology and neurological disorders in an aged companion animal.

In another aspect, there are provided animal treats (such as dog treats) for improving learning ability of aged companion animals, comprising any of the compositions described herein. For example, in some embodiments, there is provided an animal treat for improving learning ability of an aged companion animal comprising a carnitine and a lipoic acid, wherein the amount of carnitine per serving is about 100-500 mg, wherein the amount of lipoic acid per serving is about 50-250 mg, and wherein the animal treat further comprises one or more of meat by-product, poultry by-product, meat meal, bone meal, chicken or poultry liver digest, chicken or poultry viscera digest, brewer rice, and brewer dry yeast. In some embodiments, the animal treat further comprises biotin. In some embodiments, the amount of biotin per serving size is about 1 μg -1 mg, including for example about any of 1 μg-5 μg, 5 μg-10 μg, 10 μg-50 μg, 50 μg-100 μg, 100 μg-500 μg, or 500 μg-1 mg. In some embodiments, the weight ratio of the carnitine and the lipoic acid in the animal treat is about any of 5:1, 4:1, 3:1, 2:1, 1:1, 1:2, 1:3, 1:4, or 1:5. In some embodiments, the animal treat comprises carnitine and lipoic acid in a synergistic ratio. In some embodiments, the animal treat comprises less than about any of 5%, 3%, 2%, 1%, 0.5%, or 0.1% antioxidant other than carnitine and lipoic acid.

Also provided herein are kits for improving learning ability in aged companion animals, comprising carnitine, lipoic acid, and an instruction on use of the carnitine and lipoic acid for improving learning ability of the aged animal.

It is to be understood that one, some, or all of the properties of the various embodiments described herein may be combined to form other embodiments of the present invention.

### BRIEF DESCRIPTION OF THE FIGURES

FIG. 1 is a schematic diagram showing the test configuration of a modified Wisconsin General Test apparatus used in the landmark discrimination task. In Land-0, the dog is presented with two coasters, one of which is associated with a distinctive landmark. In Land-1, the landmark is moved 1 centimeter from the coaster.

FIG. 2 shows the protocol used in the delayed-non-matching-to-position (DNMP) task. The DNMP task uses all three locations of the modified test apparatus for food, which means that the sample block presentation can occur at the left, center or right food well. If the sample block presentation is to the left or the right, on the test condition, the correct object could be placed at either the center (center correct) or opposite well (center non-option). If the sample block presentation is to the center on the test trial, then the correct response can be to either the left or right well, with the center incorrect. Because the sample presentation always allows two different possible correct locations, the subject can solve the problem only by remembering the former location of the sample as the incorrect response.

FIG. 3 is a graph showing the errors-to-criterion plotted as a function of treatment Group (Control vs lipoic acid (LA, or lip) and ALCAR) and task (Land-0 and Land-1).

FIG. 4A and FIG. 4B show performance as a function of delay for each of the two groups over the first (FIG. 4A, days 1-7) and second (FIG. 4B, days 8-14) test weeks.

### DETAILED DESCRIPTION OF THE INVENTION

The present invention is based on the observation that the combination of an effective amount of a carnitine (specifically, acetyl-L-carnitine, or ALCAR) and an effective amount of a lipoic acid significantly improved the learning abilities of aged companion animals (such as dogs). Specifically, landmark tests were utilized to study the effects of ALCAR and lipoic acid on the learning ability of aged dogs. We found that aged dogs fed with specific dosages of ALCAR and lipoic acid showed significant improvement on both egocentric visuospatial learning and allocentric visuospatial learning within days after the animal treatment. By contrast, we did not observe an effect of the test compounds on the dogs' short term memory, suggesting that decline in learning ability is particularly responsive to and reversible by the administration of a combination of effective amounts of carnitine and lipoic acid.

Accordingly, the present invention in one aspect provides methods of improving learning ability of aged companion animals, comprising administering to the animal an effective amount of a carnitine (such as alkanoyl-L-carnitine, for example ALCAR) and an effective amount of a lipoic acid.

In another aspect of the present invention, there are provided companion animal treats comprising carnitine (such as alkanoyl-L-carnitine, for example ALCAR) and lipoic acid.

In another aspect of the present invention, there are provided kits for improving learning ability of aged companion animals (such as aged dogs), comprising composition(s) comprising carnitine, lipoic acid and instructions on use of the composition(s) for improving learning ability of aged companion animals.

It is understood that aspect and embodiments of the invention described herein include “consisting” and/or “consisting essentially of” aspects and embodiments.

Reference to “about” a value or parameter herein includes (and describes) embodiments that are directed to that value or parameter per se. For example, description referring to “about X” includes description of “X.”

As used herein and in the appended claims, the singular forms “a,” “or,” and “the” include plural referents unless the context clearly indicates otherwise.

### Methods of Improving Learning Ability of Aged Companion Animals

The present invention provides methods of improving learning ability of an aged companion animal, comprising administering to the aged companion animal an effective amount of a carnitine and an effective amount of a lipoic acid, wherein the amount of carnitine is at least about 8 mg/kg body weight/day and wherein the amount of lipoic acid is at least about 4 mg/kg body weight/day.

The term “companion animal” used herein refers to any non-human companion animal susceptible to or suffering from a decline in learning in aged years. Suitable companion animals include, but are not limited to, avian, bovine, canine, equine, feline, hircine, ovine, and porcine companion animals, and are particularly useful for companion animals such as canines and felines, including dogs and cats.

Dogs that can benefit from methods of the present invention include, but are not limited to, dogs of small breed, dogs of medium breed, dogs of large breed, and dogs of giant breed. In some embodiments, the dog is a domestic dog. In some embodiments, the dog is a beagle dog (such as a beagle dog model used for cognitive functional studies). The invention is particularly useful for dogs whose functions require a high level of cognitive ability, including for example working dogs (such as police dogs and guard dogs), hunting dogs, herding dogs (including sheepdogs), guide dogs, show dogs, and search and rescue dogs.

In some embodiments, the companion animal is a cat. In some embodiments, the companion animal is a horse (such as a race horse).

In some embodiments, the companion animal is a circus animal, including for example, an elephant, a dolphin, a lion, a tiger, a bear, or a horse.

In some embodiments, the companion animal is at least about any of 6, 7, 8, 9, 10, 20, or 30 years old. In some embodiments, the companion animal is a dog of small breed that is over about 8 years of age. In some embodiments, the companion animal is a dog of medium breed that is over about 7 years of age. In some embodiments, the companion animal is a dog of large breed that is over 6 years of age. In some embodiments, the companion animal is a dog of giant breed that is over 5 years of age.

In some embodiments, the companion animal is in need of improving learning ability. In some embodiments, the companion animal shows one or more signs associated with decline in learning ability. Signs associated with decline in learning ability are further discussed below.

“Learning ability” used herein includes, but are not limited to, visuospatial function (such as egocentric visuospatial function and allocentric visuospatial function), color discrimination learning, size discrimination learning, and any other functions that rely on plastic changes in neuronal transmission and/or establishment of neuronal synapses.

Improvement of learning ability can be evaluated, for example, based on one or more of the following: 1) time taken to learn a new task; 2) success rate at learning a new task; 3) error rate in carrying out a task; 4) ability to follow instruction(s); and 5) responsiveness to instruction(s). “Methods of improving learning ability” used herein include, but are not limited to, methods of enhancing learning ability, methods of reversing decline of learning ability, methods of preventing or delaying further decline of learning ability, methods of retarding decline of learning ability, and methods of reducing one or more signs associated with the decline of learning ability.

Accordingly, in some embodiments, there is provided a method of enhancing learning ability of an aged companion animal, comprising administering to the aged companion animal an effective amount of a carnitine and an effective amount of a lipoic acid, wherein the amount of carnitine is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day). In some embodiments, there is provided a method of reversing decline of learning ability of an aged companion animal, comprising administering to the aged companion animal an effective amount of a carnitine and an effective amount of a lipoic acid, wherein the amount of carnitine is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day).

In some embodiments, there is provided a method of preventing or delaying further decline of learning ability of an aged companion animal, comprising administering to the aged companion animal an effective amount of a carnitine and an effective amount of a lipoic acid, wherein the amount of carnitine is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day). In some embodiments, there is provided a method of retarding decline of learning ability of an aged companion animal, comprising administering to the aged companion animal an effective amount of a carnitine and an effective amount of a lipoic acid, wherein the amount of carnitine is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day).

In some embodiments, the invention provides methods of reducing one or more signs associated with decline of learning ability, comprising administering to the aged companion animal an effective amount of a carnitine and an effective amount of a lipoic acid, wherein the amount of carnitine is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day). Examples of such signs include, for example, lack of alertness, lack of attention, lack of vitality, lack of exploratory behavior, disorientation, sleep disturbance, housetraining deficits, restless, and symptoms of age-related neurological disorders.

Improvement of learning ability can be manifested in the aged companion animal in a number of ways. For example, improvement of learning ability can be manifested in increased vitality, increased perception and awareness, increased exploratory activity, improved quality of life of aged companion animals and/or companion animal owners, improved companionship of the aged companion animal, increased ability to respond to tasks, other animals, and people, treatment of dementia, treatment age-related deterioration of mental function, and prevention, retardation, or delay of age-related neuropathology and neurological disorders. The methods of the present invention are therefore useful for improving any one or more of the above manifestations.

In some embodiments, the improvement of learning ability of the aged companion animal can be observed within less than about one month, including for example less than about any of three weeks, two weeks, one week, 6 days, 5 days, 4 days, 3 days, 2 days, or one day. In some embodiments, the improvement of learning ability of the aged companion animal can be observed within any of about 2, 3, 4, 5, 8, 10, 12, 18, or 24 hours.

The amount of carnitine administered to the companion animal is at least about 8 mg/kg body weight/day, including for example about 8-275 mg/kg body weight/day, about 8-250 mg/kg body weight/day, about 8-225 mg/kg body weight/day, about 8-200 mg/kg body weight/day, about 8-175 mg/kg body weight/day, about 8-150 mg/kg body weight/day, about 8-125 mg/kg body weight/day, about 8-100 mg/kg body weight/day, about 8-75 mg/kg body weight/day, about 8-50 mg/kg body weight/day.

In some embodiments, the amount of carnitine administered to the companion animal is in the range of about 8-40 mg/kg body weight/day, including for example about 10-35 mg/kg body weight/day, about 15-30 mg/kg body weight/day, about 20-28 mg/kg body weight/day, or about 27.5 mg/kg body weight/day. In some embodiments, the amount of carnitine is about any of 8-10 mg/kg body weight/day, 10-15 mg/kg body weight/day, 15-20 mg/kg body weight/day, 20-25 mg/kg body weight/day, 25-30 mg/kg body weight/day, 30-35 mg/kg body weight/day, or 35-40 mg/kg body weight/day. In some embodiments, the amount to carnitine administered to the companion animal is at least about 20 mg/kg body weight/day, including for example at least about any of 25, 30, 40, 50, 60, 70, 80, or 100 mg/kg body weight/day.

“Carnitines” used herein includes, but are not limited to, L-carnitine, L-carnitine derivatives, and esters or salts of L-carnitine or L-carnitine derivatives. In some embodiments, the carnitine is L-carnitine. In some embodiments, the carnitine is an alkanoyl-L-carnitine (for example an alkanoyl-L-carnitine with a straight or branched alkanoyl group with 2-6 carbons). Suitable alkanoyl-L-carnitines include, but are not limited to, acetyl-L-carnitine, propionyl-L-carnitine, butyryl-L-carnitine, valeryl-L-carnitine, and isovaleryl-L-carnitine. Suitable salts of L-carnitine and L-carnitine derivatives include, but are not limited to, chloride, bromide, orotate, citrate, magnesium citrate, phosphate, fumarate, magnesium fumarate, lactate, maleate, oxalate, pamoate, sulphate, glucose phosphate, tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate. In some embodiments, a mixture of two or more different types of carnitines is administered to the aged companion animal.

Other examples of carnitine and method of making thereof are described in, for example, U.S. Pat. No. 4,766,222 (acyl-derivatives of carnitine); U.S. Pat. No. 4,673,534 (carnitine salts particularly suitable for oral use); U.S. Pat. No. 4,743,621 (ester of acetyl carnitine); U.S. Pat. No. 4,593,043 (mercapto acyl-carnitines); U.S. Pat. No. 4,590,209 (alkoxy-acyl carnitines); U.S. Pat. No. 4,567,200 (Esters of mercapto acyl-carnitines); U.S. Pat. No. 4,032,641 (nicotinoyl carnitine derivatives); U.S. Pat. No. 4,551,477 (esters of alkoxy-acylderivatives of carnitine); U.S. Pat. No. 4,401,827 (acyl-derivatives of carnitine); U.S. Pat. No. 5,260,464 (carnitine derivatives); U.S. Pat. No. 4,859,698 (acyl-derivatives of carnitine); U.S. Pat. No. 4,692,543 (optically-active di-[3-chloro-2-oxy-propyltrimethylammonium]-tartrate); U.S. Pat. No. 5,258,552 (N-alkylamides of d(+)-carnitine having antibacterial activity).

In some embodiments, the carnitine is L-carnitine. In some embodiments, the carnitine is acetyl-L-carnitine (ALCAR). ALCAR is superior to L-carnitine in several aspects. For example, ALCAR crosses the blood-brain barrier more readily, is more readily taken up by cells, and can function as a donor of the acetyl group to choline to produce the neurotransmitter acetylcholine. ALCAR is also more effective than L-carnitine in neuroprotection. In addition, ALCAR can reverse age-related decrease in cardiolipin, age-associated decrease in mitochondria DNA transcription, and decreased membrane potential. ALCAR is therefore particularly suitable for methods described herein.

The amount of lipoic acid administered to the companion animal is at least about 4 mg/kg body weight/day, including for example about 4-110 mg/kg body weight/day, about 4-100 mg/kg body weight/day, about 4-90 mg/kg body weight/day, about 4-80 mg/kg body weight/day, about 4-70 mg/kg body weight/day, about 4-60 mg/kg body weight/day, about 4-50 mg/kg body weight/day, about 4-40 mg/kg body weight/day, about 4-30 mg/kg body weight/day.

In some embodiments, the amount of lipoic acid is in the range of about 4-20 mg/kg body weight/day, including for example about any of about 8-15 mg/kg body weight/day, about 10-15 mg/kg body weight/day, or about 11 mg/kg body weight/day. In some embodiments, the amount of lipoic acid is any of about 4-10 mg/kg body weight/day, about 10-15 mg/kg body weight/day, and about 15-20 mg/kg body weight/day. In some embodiments, the amount of lipoic acid is at least about 10 mg/kg body weight/day, including for example at least about any of about 20, 30, 40, or 50 mg/kg body weight/day.

As used herein, “lipoic acid” includes α-lipoic acid, and can be provided in various forms including recemic mixtures, salts, esters, amides, and other lipoate derivatives (including metabolites of lipoic acid). Examples of lipoate derivatives are provided in U.S. Pat. No. 5,621,117. Suitable derivatives of lipoic acid include, for example, dihydrolipoic acid, lipoamide, lipoyllysine, bisnorlipoic acid, and tetranorlipoic acid. In some embodiments, a mixture of two or more different types of lipoic acid is administered.

Manufactured lipoic acid typically occurs in a racemic form comprising equal amounts of R- and L-forms. Only R-lipoic acid occurs naturally. In some embodiments, the lipoic acid used in methods and compositions of the present invention is R-lipoic acid. In some embodiments, the lipoic acid is enriched with R-lipoic acid. In some embodiments, the lipoic acid is a mixture of R-lipoic acid and S-lipoic acid having an R-enantiomer excess (EE) of at least about 40% (including for example at least about any of 50%, 60%, 70%, 80%, 90%, 95%, or 95%). The enantiomer excess (EE) is given by the following formula: EE[%]=(R−S)/(R+S)×100. R and S refer to R-lipoic acid and S-lipoic acid, respectively. The enantiomerically pure compound (EE=100%) is also referred to as a homochiral compound or a pure form of R-lipoic acid.

In some embodiments, the weight ratio of the carnitine and the lipoic acid administered to the aged companion animal is about any of 5:1, 4:1, 3:1, 2:1, 1:1, 1:2, 1:3, 1:4, or 1:5. In some embodiments, the weight ratio is about 2:1. In some embodiments, the weight ratio is about 1:1. In some embodiments, the ratio of carnitine and lipoic acid is a synergistic ratio.

In some embodiments, the administration of carnitine and lipoic acid is intermittent. In some embodiments, the frequency of the administration is regular. In some embodiments, the frequency of the administration is irregular.

In some embodiments, the carnitine and the lipoic acid are administered once daily. In some embodiments, the carnitine and the lipoic acid are administered at least about two times daily, including for example at least about any of 3, 4, 5, 6, 7, or 8 times a day. The amounts of the compounds at each administration can be equal or different, so long as a total daily dosage is administered. Furthermore, less frequent administration such as once every 2 days, once every 3 days, once every week, is also contemplated.

Carnitine and lipoic acid are preferably administered orally. Other routes of administration such as intravenous, parenteral, subcutaneous, transdermal, intranasal, rectal, or inhalation, are also contemplated.

Because both carnitine and lipoic acid are water soluble, it is sometimes desirable that both compounds be administered to an empty stomach of the aged companion animal to ensure maximum absorption. For example, in some embodiments, the carnitine and the lipoic acid are administered at least about 2, 3, 4, 5, 6, 7, 8, 9, or 10 hours after a meal (such as the main meal(s)) of the companion animal. In some embodiments, the carnitine and the lipoic acid are administered at least about any of 0.2, 0.5, 1, 2, 3, 4, 5, or 6 hours prior to the administration of a meal (such as the main meal(s)) of the companion animal. It is of course also contemplated that the carnitine and the lipoic acid be administered along with the food, or less than about any of 0.2, 0.5, 1, 2, or 3 hours before or after the administration of a meal (such as the main meal(s)) of the companion animal.

In some embodiments, the carnitine and the lipoic acid are administered simultaneously. The term “simultaneous administration,” as used herein, means that the carnitine and the lipoic acid are administered with a time separation of no more than about 15 minutes, such as no more than about any of 10, 5, 3 minutes, or no more than about 1 minute. When carnitine and lipoic acid are administered simultaneously, they can be combined in the same composition (e.g., a composition comprising both carnitine and lipoic acid) or in separate compositions (e.g., the carnitine is contained in one composition and the lipoic acid is contained in another composition). Compositions comprising a carnitine and a lipoic acid that are suitable for methods of the present invention are further described herein. In some embodiments, simultaneous administration of carnitine and lipoic acid can be combined with supplemental amounts of carnitine and/or lipoic acid.

In some embodiments, carnitine and lipoic acid are administered sequentially. The term “sequential administration” as used herein means that carnitine and lipoic acid are administered with a time separation of more than about 15 minutes, such as more than about any of 20, 30, 40, 50, 60, or more minutes. Either carnitine or lipoic acid can be administered first. Carnitine and lipoic acid are contained in separate compositions, which may be contained in the same or different packages.

The method described herein may further comprise administration of one or more additional component(s) to the aged companion animal. For example, in some embodiments, one or more members of the vitamin B complex, such as thiamine, riboflavin, nicotinic acid, pyridoxine, panthothenic acid, biotin, folic acid, cyanocobalamin, choline, inositol, and para-amino-benzoic acid, are also administered to the aged companion animal.

In some embodiments, the method further comprises administering biotin to the aged companion animal. Biotin is important for maintaining optimal levels of metabolites utilized for energy production in the mitochondria. It plays essential roles in the biosynthesis of macronutrients such as fatty acids, gluconeogenesis, and metabolism of critical branched-chain amino acids (e.g., L-leucine, L-isoleucine, and L-valine). Biotin also is integral to the de novo synthesis of purine nucleosides.

The molecular structure of biotin is similar to that of a-lipoic acid. As a consequence of this structural similarity, an excess of α-lipoic acid could compete with or displace biotin from its binding partners. Accordingly, in some embodiments of the present invention, biotin is co-administered (either simultaneously or sequentially) with carnitine and lipoic acid to offset a potential imbalance with lipoic acid. Suitable amount of biotin includes, for example, 0.05-100 μg/kg body weight/day, including for example any of about 0.05-0.1 μg/kg body weight/day, about 0.1-0.5 μg/kg body weight/day, about 0.5-1 μg/kg body weight/day, about 1-5 μg/kg body weight/day, about 5-10 μg/kg body weight/day, about 10-50 μg/kg body weight/day, and about 50-100 μg/kg body weight/day.

### Methods of Administering a Composition Comprising a Carnitine and a Lipoic Acid

As discussed above, the carnitine and the lipoic acid can be administered in a single composition. Thus, the methods described herein may comprise administering to the aged companion animal a composition comprising (including consisting of or consisting essentially of) an effective amount of a carnitine and an effective amount of a lipoic acid, wherein the amount of carnitine is at least about 8 mg/kg body weight/day (including for example about 8-40 mg/kg body weight/day or at least about 20 mg/kg body weight/day) and wherein the amount of lipoic acid is at least about 4 mg/kg body weight/day (including for example about 4-20 mg/kg body weight/day or at least about 10 mg/kg body weight/day).

In some embodiments, the composition comprises at least about 2% (w/w) (including for example at least about any of 5%, 10%, 20%, 30%, 40%, or 50%) carnitine and at least about 1% (w/w) (including for example at least about any of 2%, 3%, 5%, 10%, 20%, or 25%) lipoic acid. In some embodiments, the weight ratio of the carnitine and the lipoic acid in the composition is about any of 5:1, 4:1, 3:1, 2:1, 1:1, 1:2, 1:3, 1:4, or 1:5. In some embodiments, the composition comprises a synergistic ratio of carnitine and lipoic acid.

In some embodiments, the composition further comprises one or more members of the vitamin B complex, including, but are not limited to, thiamine, riboflavin, nicotinic acid, pyridoxine, panthothenic acid, biotin, folic acid, cyanocobalamin, choline, inositol, and para-amino-benzoic acid. In some embodiments, the composition further comprises biotin (for example at least about any of 0.001%, 0.01%, 0.02%, 0.05%, 0.1%, 0.5%, 1%, 2%, or 5% of biotin).

In some embodiments, the composition comprises additional antioxidants, such as vitamin C and vitamin E. In some embodiments, the weight ratio of carnitine to vitamin E in the composition is at least about 0.5:1, such at least about any of 1:1, 2:1, 5:1, 10:1, 15:1, 20:1, 25:1, or 30:1. In some embodiments, the weight ratio of lipoic acid to vitamin E in the composition is at least about 0.25:1, such as at least about any of 0.5:1, 1:1, 2:1, 5:1, 10:1, or 15:1. In some embodiments, the weight ratio of carnitine to vitamin C in the composition is at least about 4:1, including for example at least about any of 5:1, 6:1, 7:1, 8:1, 9:1, or 10:1. In some embodiments, the weight ratio of lipoic acid to vitamin C in the composition is at least about 2:1, including for example at least about any of 3:1, 4:1, or 5:1. In some embodiments, the composition comprises no greater than about any of 5%, 3%, 2%, 1%, or 0.5% antioxidant(s) other than carnitine and lipoic acid.

The composition used in methods of the present invention may further comprise any one or more of the following: meat by-product, poultry by-product, meat meal, bone meal, chicken or poultry liver digest, chicken or poultry viscera digest, brewer rice, and brewer dry yeast. Other ingredients commonly used for pet food can also be included in the composition described herein. For example, the composition may comprise one or more of the following: soybean meal, flakes or pellets, vegetable meal, flakes or pellets, animal waste products and processed animal waste, dried poultry waste, dried poultry litter, dried ruminant waste, dried swine waste, undried processed animal waste products, processed animal waste derivatives, raw leather and leather hydrolysate, silage additive products, clay, ash, bagasse (sugar cane pulp), blood and blood albumin, bolls, bones, cannery residue, carcass meat trimmings and carcass residue, charcoal, corn cobs and ears, cracklings, diluent, distillation solubles, dust, feathers, fodder, grit, hulls and husks, sterols, tallow, viscera, animal serum, serum albumin, serum globulin, hair, egg shells and egg shell meal, unborn calf carcasses, animal digest, bone marrow, animal plasma, ansiled paunch, fish by-products, fish meal, hydrolysates (digests) of fish and shellfish, lupin meal, restaurant food waste, salvage pet food, distressed pet food, light and broken grains, agricultural seeds, weed seeds, hulls, chaff, joints, straw, elevator or mill dust, sand and dirt, urea, anhydrous ammonia, formaldehyde, reed-sedge peat. In some embodiments, the composition comprises at least one ingredient that is unsuitable for consumption by human.

In some embodiments, the composition further comprises any one or more of the following: chicken meal, rice flour, wheat flour, corn syrup, water, glycerin, molasses, tapioca starch, gelatin, natural flavor, chicken fat (such as chicken fat preserved with mixed tocopherols and citric acid), salt, phosphoric acid, L-ascorbyl-2-polyphosphate, potassium sorbate, mono and diglycerides, vitamin E supplement, minerals (such as zinc proteinate, iron, proteinate, copper proteinate, manganese proteinate, cobalt proteinate), calcium propionate, garlic powder, and biotin supplement.

The compositions used in methods of the present invention may incorporate meat or animal derived material (such as beef, chicken, turkey, lamb, pork, fish, blood plasma, marrowbone, etc.). Alternatively, the compositions may be meat free, and include for example meat substitute such as soya, maize, gluten, or a soya product. The composition may contain additional protein sources such as soya protein concentrate, milk proteins, gluten, etc. The composition may also include a starch source such as one or more grains (for example wheat, corn, rice, oats, barley, etc.).

In some embodiments, the weight percentage of protein in the composition is about 21% or more. In some embodiments, the weight percentage of fat in the composition is about 4.5% or more. In some embodiments, the weight percentage of fat in the composition is about 7% or less. In some embodiments, the weight percentage of fiber in the composition is about 2% or more.

Compositions described herein can be made in accordance with customary processing techniques for veterinary products. In some embodiments, the composition is formulated as nutritional supplements, for example in the forms of pills, tablets, capsules, etc. In some embodiments, the composition is an animal food (such as balanced animal food) containing an effective amount of a carnitine and a lipoic acid. In some embodiments, the composition is a liquid beverage or a dry beverage mix. The composition may also be formulated as an animal treat. Treats for companion animals comprising a carnitine and a lipoic acid are further described herein.

### Animal Treats for Improving Learning Ability of Aged Companion Animals

Another aspect of the present invention provides animal treats for improving learning ability of aged companion animals. For example, in some embodiments, there is provided an animal treat for improving learning ability of an aged companion animal comprising a carnitine and a lipoic acid, wherein the amount of carnitine per serving is about 100-500 mg, wherein the amount of lipoic acid per serving is about 50-250 mg, and wherein the animal treat further comprises any one or more of the following: meat by-product, poultry by-product, meat meal, bone meal, chicken or poultry liver digest, chicken or poultry viscera digest, brewer rice, and brewer dry yeast. In some embodiments, the animal treat further comprises biotin. In some embodiments, the animal treat comprises at least one ingredient that is unsuitable for consumption by human. In some embodiments, the amount of biotin per serving size is about 1 μg-1 mg, including for example about any of 1 μg-5 μg, 5 μg-10 μg, 10 μg-50 μg, 50 μg-100 μg, 100 μg-500 μg, or 500 μg-1 mg.

In some embodiments, the amount of carnitine per serving is about any of 100-200 mg, 200-300 mg, 300-400 mg, or 400-500 mg. In some embodiments, the amount of lipoic acid per serving is about any of 50-100 mg, 100-150 mg, 150-200 mg, or 200-250 mg.

In some embodiments, the animal treat comprises at least about 2% (w/w) (including for example at least about any of 5%, 10%, 20%, 30%, 40%, or 50%) carnitine and at least about 1% (w/w) (including for example at least about any of 2%, 3%, 5%, 10%, 20%, or 25%) lipoic acid. In some embodiments, the weight ratio of the carnitine and the lipoic acid in the animal treat is about any of 5:1, 4:1, 3:1, 2:1, 1:1, 1:2, 1:3, 1:4, or 1:5. In some embodiments, the animal treat comprises carnitine and lipoic acid in a synergistic ratio.

In some embodiments, the animal treat further comprises biotin (for example at least about any of 0.001%, 0.01%, 0.02%, 0.05%, 0.1%, 0.5%, 1%, 2%, or 5% of biotin).

In some embodiments, the animal treat comprises one or more additional antioxidants, such as vitamin C and vitamin E. In some embodiments, the weight ratio of carnitine to vitamin E in the animal treat is at least about 0.5:1, such at least about any of 1:1, 2:1, 5:1, 10:1, 15:1, 20:1, 25:1, or 30:1. In some embodiments, the weight ratio of lipoic acid to vitamin E in the animal treat is at least about 0.25:1, such as at least about 0.5:1, 1:1, 2:1, 5:1, 10:1, or 15:1. In some embodiments, the weight ratio of carnitine to vitamin C in the animal treat is at least about 4:1, including for example at least about any of 5:1, 6:1, 7:1, 8:1, 9:1, or 10:1. In some embodiments, the weight ratio of lipoic acid to vitamin C in the animal treat is at least about 2:1, including for example at least about any of 3:1, 4:1, or 5:1. In some embodiments, the animal treat comprises no greater than about any of 5%, 3%, 2%, 1%, or 0.5% antioxidant other than carnitine and lipoic acid.

In some embodiments, the animal treat further comprises any one or more of the following in addition to carnitine and lipoic acid: chicken meal, rice flour, wheat flour, corn syrup, water, glycerin, molasses, tapioca starch, gelatin, natural flavor, chicken fat (such as chicken fat preserved with mixed tocopherols and citric acid), salt, phosphoric acid, L-ascorbyl-2-polyphosphate, potassium sorbate, mono and diglycerides, vitamin E supplement, minerals (such as zinc proteinate, iron, proteinate, copper proteinate, manganese proteinate, cobalt proteinate), calcium propionate, garlic powder, and biotin supplement.

In some embodiments, the animal treat has a formulation as shown in Table 2.

In some embodiments, the weight percentage of protein in the animal treat is about 21% or more. In some embodiments, the weight percentage of fat in the animal treat is about 4.5% or more. In some embodiments, the weight percentage of fat in the animal treat is about 7% or less. In some embodiments, the weight percentage of fiber in the animal treat is about 2% or more.

The animal treats described herein can be, for example, in the forms of snack bars, biscuits, chews, and sweet products. The sizes of the treats can vary. For example, each piece of the treat can be about any of 1, 2, 3, 4, 5, 6, 7, 8, 9, or 10 grams. The animal treats can be nutritional or non-nutritional. Carnitine and lipoic acid can be present in a coating on the surface of the animal treat, admixed with other ingredients in the animal treat, wrapped inside the animal treat, or a combination thereof.

Also provided are methods of improving learning ability of aged companion animals, comprising administering to the aged companion animal an animal treat described herein.

### Kits for Methods of the Present Invention

Also provided are kits comprising a carnitine and a lipoic acid suitable for methods described herein. The kits may comprise carnitine and lipoic acid in the same or different compositions. Carnitine and lipoic acid, when in different compositions, can be in the same container or in different containers. In some embodiments when carnitine and lipoic acid are in separate containers, they can be in a single package or in separate containers in a virtual package.

The term “single package” means that the components of a kit are physically associated in or with one or more containers and considered a unit for manufacture, distribution, sale, or use. Containers include, but are not limited to, bags, boxes, bottles, shrink wrap packages, cans, foil-wrapped packages, plastic wrapped packages, stapled or otherwise affixed components, or combinations thereof. A single package may be containers of individual compound physically associated such that they are considered a unit for manufacture, distribution, sale, and use. The term “virtual package” means that the components of a kit are associated by directions on one or more physical or virtual kit components instructing the user how to obtain the other components, e.g., in a bag containing one component and directions instructing the user to go to a website to obtain information about the other component and/or instructions on how to use the kit.

The kits may further comprise an instruction for methods of the present invention. The instructions included with the kit generally contain information as to the components and their administration to a companion animal. For example, the instruction may comprise instruction on how to use the components for methods described herein and/or instructions on how to combine carnitine and lipoic acid to produce a composition useful for methods described herein. Means for communicating the instructions include, for example, written documents, digital storage media, optical storage media, audio presentation, or visual display containing such information or instructions. In some embodiments, the communication means is a displayed website or a brochure, product label, package insert, advertisement, or visual display containing information or instruction on use of methods described herein.

Although the foregoing invention has been described in some detail by way of illustration and example for purposes of clarity of understanding, it is apparent to those skilled in the art that certain minor changes and modifications will be practiced.

### EXAMPLES

### Example 1

This example demonstrates the effect of ALCAR and lipoic acid on the learning abilities of aged dogs based on three different cognitive endpoints: acquisition of a landmark discrimination learning task, referred to as Land-0; acquisition of a second landmark discrimination learning task, referred to as Land-1; and performance on a test of visuospatial working memory, a variable-delay non-matching-to-position task (DNMP).

The landmark discrimination task was first described for use with dogs by Milgram et al. (1999), Learning and Memory, 6, 54-61, and consisted of a minimum of two phases. In the first, referred to as Land-0, the subjects were trained to discriminate between two coasters; the correct one had a landmark (a yellow wooden peg) attached to the center, and assessed general discrimination learning ability. In the second task, Land-1, the peg was 1 cm away from the coaster, and the subjects were required to approach the object closest to the landmark. Land-1 provides a measure of allocentric spatial ability, which involves the utilization of the external landmark as a spatial reference.

The DNMP task was first described by Head et al. (1995), Behavioral Neuroscience, 109, 851-858; and Chan et al. (2002), Behavioral Neuroscience, 116:443-54. The present study used variable-delay DNMP protocol, in which the dog's ability to remember the location of a reward was tested over a short (5 S), medium (55 S), and long (105 S) time interval. The DNMP task was included in order to assess the effects of ALCAR and lipoic acid on visuospatial memory.

### Methods

### Administration of ALCAR and Lipoic Acid (LA)

A matched group, parallel group design was used to compare the effects of the test compounds (LA and ALCAR) on a landmark discrimination learning protocol and on performance on a DNMP. Grouping was based on performance on a delayed non-matching to position task (DNMP) over 5 days. The protocol started with a 5-day wash-in phase in which the subjects received their full dose but were not cognitively tested. Subjects were maintained on the test compounds during the entire testing period. Cognitive testing occurred 2-3 hours after dosing. The landmark discrimination protocol assessed accuracy and speed of learning on two tasks, Land-0 and Land-1, over a maximum of 55 days. After completing the landmark discrimination protocol, the subjects were switched to a visuospatial working memory task for a 14-day test.

The subjects were random-source beagle dogs of both sexes between 7.6 and 8.8 years of age from the colony of CanCog Technologies, Inc. (Ontario, Canada). All subjects were test-sophisticated and had previously successfully learned a DNMP task. Prior to the start of this study, all subjects were administered the DNMP task over 5 consecutive sessions. Group selection and assignment was based on performance at baseline on a DNMP task, sex and age. The animals, group assignments and relevant grouping variables are shown in Table 1.

TABLE 1
Methlylcellulose (control)

| | | date | age | Baseline |
|---|---|---|---|---|
| Subject | sex | Of birth | at start | (DNMP) |
| Chilli | F | 21/07/1998 | 7.8 | 0.73889 |
| Banjo | M | 21/06/1997 | 8.8 | 0.675 |
| Randy | M | 21/06/1998 | 7.8 | 0.74444 |
| Donnie | M | 21/06/1998 | 7.8 | 0.77778 |
| Cherry | F | 8/10/1997 | 8.5 | 0.73611 |
| Waggles | F | 11/07/1998 | 7.8 | 0.725 |
| Mean | | | 8.1 | 0.73287 |

Lipoic Acid and Acetyl-L-Carnitine

| | | date | | |
|---|---|---|---|---|
| Subject | sex | Of birth | age | (DNMP) |
| Bubbles | F | 27/09/1998 | 7.6 | 0.72222 |
| Barry | M | 21/06/1998 | 7.8 | 0.66111 |
| Miami | F | 19/08/1997 | 8.7 | 0.73889 |
| Joey | M | 21/06/1997 | 8.8 | 0.74444 |
| Debbie | F | 20/08/1997 | 8.7 | 0.63333 |
| Steve | M | 11/07/1998 | 7.8 | 0.733333 |
| Mean | | | 8.2 | 0.70417 |

The subjects were housed in stainless steel pens, either approximately 2.5′×16′ or 5′×16′, with 2′×4′ perches. The floors were epoxy painted and heated and the exterior walls had windows near the ceiling (approximately 10′ from ground level) that allowed natural lighting. The subjects were housed in compatible groups (3 or 4 to a pen) whenever possible and were given daily outdoor exercise for approximately half an hour per day. The pens were cleaned daily with a power washer and the housing area was disinfected daily with a sanitizing agent.

Methylcellulose was used as the control compound, and the test compound consisted of a powder of alpha-lipoic acid (LA) and acetyl-1-carnitine (ALCAR). The total daily dose levels of LA was 11.0 mg/kg, and the daily dose of ALCAR was 27.5 mg/kg. The test and control compounds were given orally in capsules placed in meatballs consisting of Hill's Prescription Diet (PD) (Hill's Pet Nutrition, Inc., P.O. Box 148, Topeka, Kans. 66601). Each animal was dosed twice each day, at approximately 8:00 am and about 3:45 pm. Each capsule contained half the total daily dose (13.75 mg/kg ALCAR and 5.5 mg/kg of LA). Cognitive testing was performed 2-3 hours after morning dosing.

All phases of the study were blinded. The technicians responsible for preparing the capsules were separate from the technicians responsible for administering the capsules. A third group of technicians carried out the cognitive testing and were blinded as to the animal treatment conditions of the animals.

Prior to the start of the test phase, blood work was acquired from all of the subjects to help obtain a safety profile of administration of the test compound to canines. A second blood sample was taken on Day 35. In both instances, the samples were sent out for analysis of hematology (WBC, RBC, RBC indices, platelet, WBC differential and morphology) and clinical chemistry (albumin (A), ALP, ALT, bilirubin [total], urea, creatinine, glucose, protein [total], globulin (G), and A/G ratio).

### Tests Apparatus and Software

The testing apparatus was a wooden box (approximately 3′×5′), which was originally described by Milgram et al (1994). The box is a modified version of the Wisconsin General Test Apparatus, commonly used in assessing cognitive function in nonhuman primates. The front side has three height-adjustable gates through which the dog responds. The experimenter is separated from the dog by a partition containing a one-way mirror and a hinged door that when opened allows the tray to be presented to the dog. The tray contains one medial and two lateral food wells which are used for all but the motor task and four-choice oddity task. The two lateral food wells are used in the landmark discrimination test protocol.

All cognitive testing used our DogCog computer program (CanCog Technologies Inc., proprietary computer program for internal use at CanCog. www.cancog.com). The program was under administrator control, which prevented the cognitive test technicians from modifying any of the test parameters. The DogCog program randomly calculated all test sequences, and controlled all timing, inter-trial intervals, inspection intervals, and delays. It was also used to record each animal's responses and latencies and assured that the same sequence of correct locations was used for every animal on each test session. Prior to each trial, the computer emitted a tone that served as a cue for the dog and instructed the experimenter to present the food tray. Each trial started when the experimenter pressed a key and simultaneously presented the tray to the subject. The dogs' response selection was recorded by a key press, which also indicated the end of the trial and signaled the beginning of the inter-trial interval.

### Training on Landmark Discrimination Test Protocol

This task was aimed at training subjects to approach one of two objects (white coasters) based on their proximity to an external landmark (yellow peg). Training occurred in two phases. First, the dogs were trained to approach one of the two white coasters covering one of the three food wells. The correct response was dictated by the location of a yellow wooden peg, measuring 2 cm×2 cm×9 cm, which served as the landmark. On the first task, called Land-0, the landmark peg was attached to the center of one of the two coasters. The dogs participated in 10 learning trials per day. On each trial, the experimenter placed a food reward, approximately 1 gram of wet dog food (Hills PD), in either the left or right food well. The coaster with the landmark peg was then placed over the food. The door was raised and the tray was moved to approximately 25 cm from the dog for a brief inspection interval, to enable the dog to see the spatial arrangement on the tray. The tray was then presented more closely to the dog, and the dog was given up to one minute to respond by displacing one of the two food-well covers, with the correct one being the coaster with the attached landmark. The location of the landmark was determined randomly by the computer, with the constraint that each side was correct on half of the trials of each test session.

Each dog was first given a maximum of 10 test sessions (totaling 100 trials) to learn to respond to stimulus associated with the landmark for L-0. Dogs received 10 trials per day, with an inter-trial interval of 30 seconds. Test sessions were conducted once a day. A partial-correction procedure was used in which the dog was permitted to correct its response after making an error once each session. Testing was completed when the dog successfully passed the two-stage criteria. To pass the first stage, the dog was required to respond correctly on at least 9 of 10 trials on one day, or on 8 of 10 trials over 2 consecutive days, and to have no response failures. A response failure occurred if a dog failed to respond within the allotted 60 seconds. In such instances, dogs were assigned a score of 0.5, which is what their score would have been had they responded randomly. The second-stage criterion was met when the subject responded correctly on at least 70% of the next 30 trials (three consecutive sessions), again with no response failures. Subjects were given an extra day of testing to complete the second-stage criterion: an average score of 70% over the three last test days was required to pass the second stage.

If a dog did not pass within the first 10 sessions, it was given a program of remedial training, which consisted of 5 additional training days, with 15 trials per day. At the start of the remedial training, dogs were presented with a single reward stimulus on the majority of the trials. With continued testing, more paired stimulus presentations were given. After completing the remedial learning phase, the dogs received up to a maximum of 10 additional training sessions using the original protocol. At the completion of testing, they were assigned an errors-to-criterion learning score that was based on either the total number of errors made to successfully pass the task, or the total number within the 20 training trials (with the remedial training excluded).

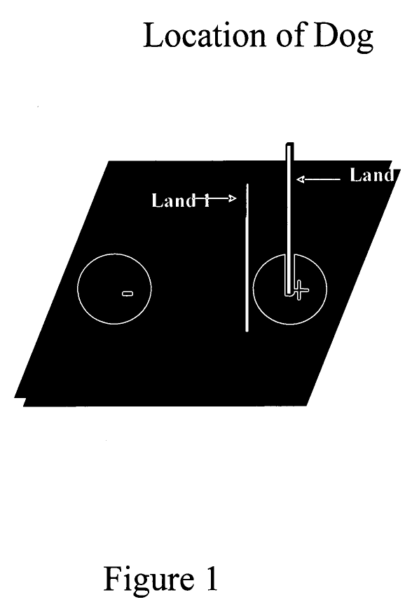

Training on Land-1 started on the day after each dog successfully met criteria on Land-0. On the Land-1 task, the landmark peg was moved 1 cm medially and diagonally away from the edge of the coaster that had together with the peg had constituted the landmark in Land-0 (cf FIG. 1). The landmark peg was attached to the food tray by a 2-cm-wide black double-sided VELCRO strip, which was attached to both the base of the landmark and the surface of the food tray. Each dog was given up to 25 training sessions to successfully meet the same two-stage criteria used in Land-0. In all other respects, the training procedures were identical to that followed in Land-0.

### Testing on the Delayed-Non-Matching-To-Position (DNMP) Task

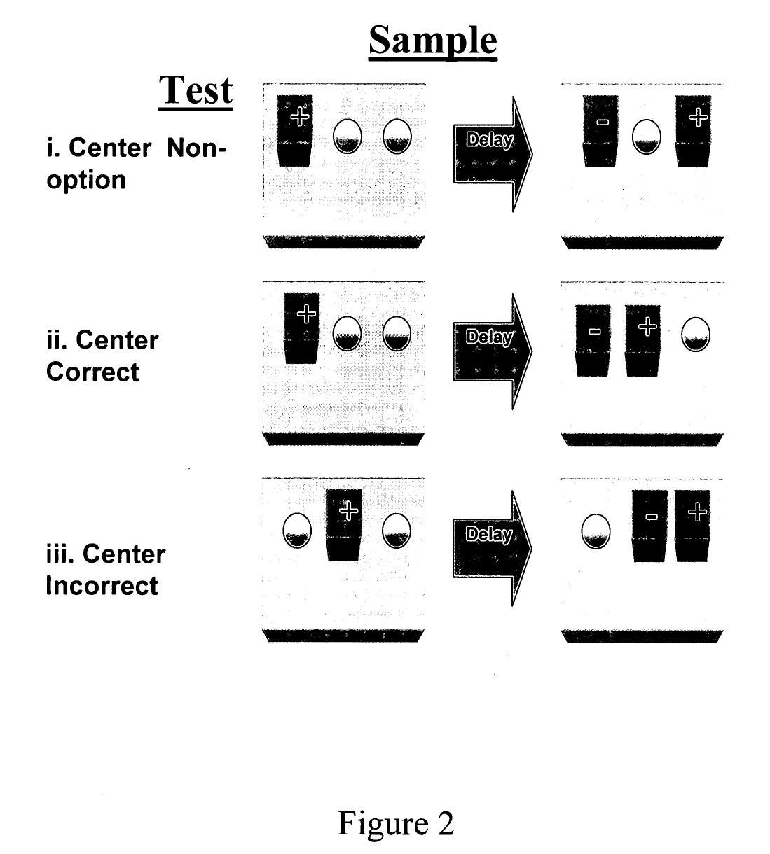

All dogs used in this study had previously been trained on the DNMP task and had had substantial practice prior to the start of this study. Two days after completing testing on Land-1, all dogs were given 14-test sessions on consecutive days on a variable-delay DNMP task. The DNMP task utilized visuospatial working memory to establish the correct location of a food reward. Each trial consisted of two presentations, a sample presentation and a test presentation. For the sample presentation, the subject was presented with a single red block covering one of the three food wells and allowed up to 60 S to displace the block and retrieve the food reward. After the sample phase, a delay interval was initiated. The test presentation followed the delay and involved presentation of two identical blocks, one of which was at the food position while the other was at a one of the other two food-well locations. Food reward was available only if the subject responded to the block at the new location (see FIG. 2). For each test session, delays of 5, 55 and 105 S were equally divided among the 18 trials; thus on each test, there were exactly 6 trials with a delay of 5 S, 6 with a delay of 55, and 6 with a delay of 105 S. The inter-trial interval was 30 S.

### Results

### Performance as a Function of Treatment on Land-0 and Land-1

Four out of six subjects in the LA+ALCAR group and two of six from the control group passed the initial Land-0 test within the first 10 training sessions. One additional control and both animals on the test compound passed after completing remedial training. The Land-1 test was more difficult. Five of six subjects in the LA+ALCAR group successfully completed the two-stage learning criterion, but only three of the six animals in the control group passed the task.

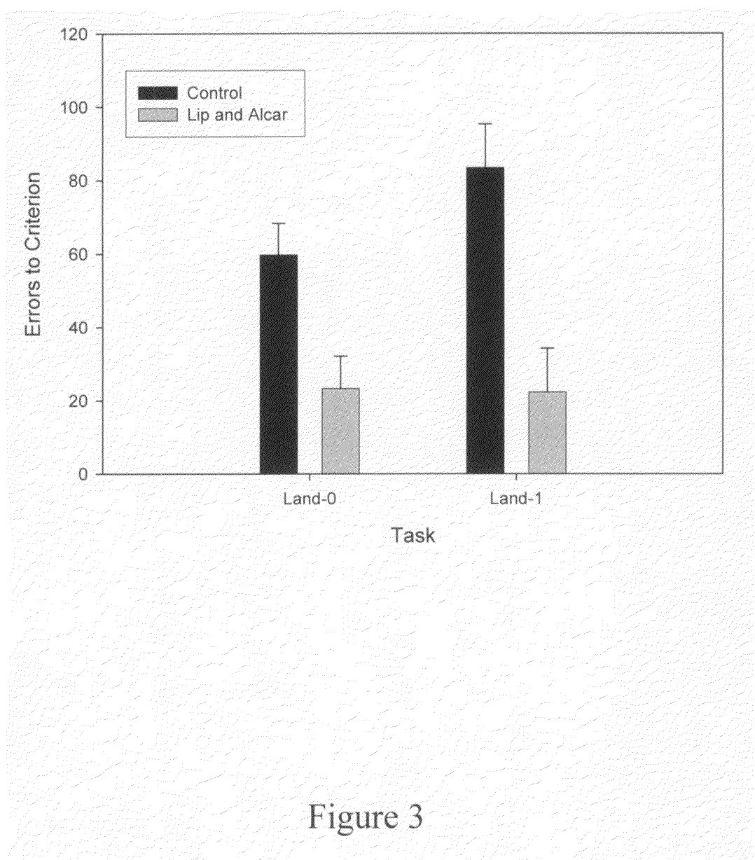

Total errors made on each of Land-0 and Land-1 were used in comparing the LA+ALCAR group with the control group. The data were first analyzed with a repeated-measure analysis of variance, with the task (Land-0 and Land-1) as a within-subject measure and with the animal treatment (test compound or control compound) as a between-subject measure. Univariate ANOVA's were then used to compare the groups on each task. The initial ANOVA revealed a highly significant treatment effect (f(1,10)=13.77; p=0.004), and a marginally significant interaction between task and treatment (F(1,10)=3.414; p=0.0945), and no other significant main effects or interactions. The separate univariate ANOVA's indicated highly significant differences on both Land-0 and Land-1 tasks (p=0.012, p=0.005, respectively). FIG. 3 illustrates that these data reflect that the LA+ALCAR group made significantly fewer errors on both tasks than did the control group. The marginally significant interaction reflects slightly improved performance on Land-1 compared to Land-0 in the LA+ALCAR group, but impaired performance in Land-1 compared to Land-0 in the Control Group.

### Performance on DNMP Test of Visuospatial Working Memory

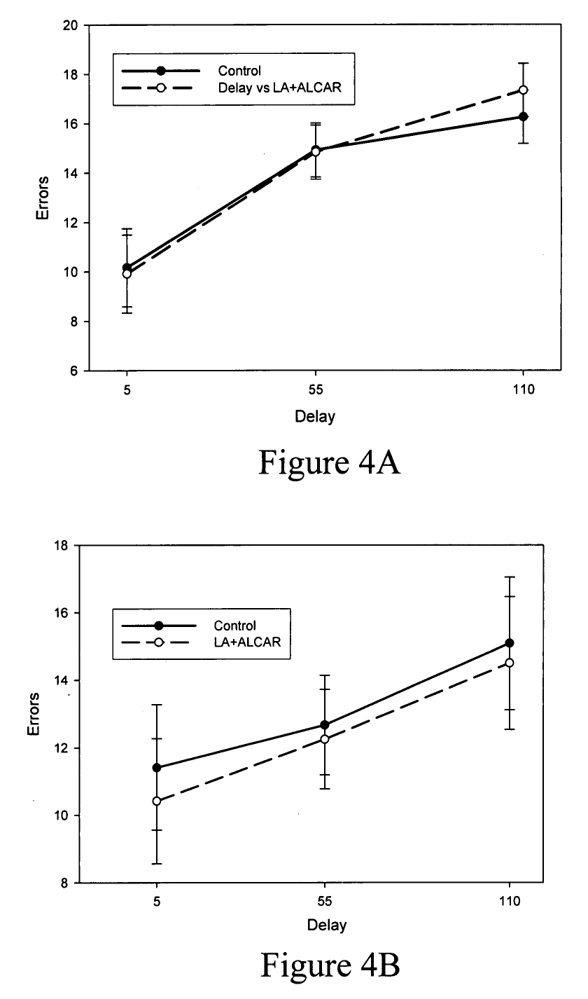

The study protocol called for comparison of the control and treatment groups over two test weeks. The groups were therefore compared with a repeated-measure ANOVA with delay (5, 55, and 105 S) and time of test (week 1 vs week 2) as within-subject variables and with treatment (control or LA+ALCAR) as a between-subject variable. The analysis revealed, as expected, a highly significant effect of delay; however, there were no other significant main effects or interactions. These results are illustrated graphically in FIGS. 4A and 4B and show that errors increased progressively as the delay interval increased. FIGS. 4A and 4B also show that the LA+ALCAR group made slightly fewer errors in the second week than the control group, but the differences did not approach statistical significance.

Although there were no group differences on treatment or control, Table 1 indicates that under baseline condition, the mean DNMP performance of the control group was about 3% higher than the baseline performance of the group treated with LA+ALCAR. To take this into consideration, the subjects' scores were converted to a percent incorrect for control, week 1, and week 2. This conversion was performed because the baseline testing was based on only 5 test sessions. These data were then reanalyzed with a repeated-measure ANOVA with time of test (baseline, week 1 and week 2) and delay as within-subject variables and with treatment as the between-subject variable. Still, there was a significant effect of delay (F(2,20)=23.72; p=0.000), but no other significant main effects or interactions.

### Discussion

These results demonstrate significant positive effects on acquisition of a landmark discrimination learning protocol of maintenance of aged dogs on an animal treatment consisting of LA+ALCAR, when compared to dogs treated with a methylcellulose control. The landmark discrimination test protocol consisted of two tasks, which were intended to assess allocentric learning ability. In the first test, Land-0, dogs were rewarded for approaching an object consisting of a coaster plus an attached wooden rod. This task depended on object discrimination learning, in which the dogs learn to associate one of two objects with a reward. By contrast, the Land-1 task constitutes an allocentric discrimination learning task because the wooden rod was physically separate from the coaster, and served as an external landmark. The group differences in learning both tasks were highly significant, with the animals in the LA+ALCAR group showing significantly more accurate learning than the controls. Furthermore, all the LA+ALCAR animals successfully passed Land-0, and 5 of 6 passed Land-1. By contrast, 3 of 6 controls failed Land-0, and 4 of 6 controls failed Land-1. Moreover, the magnitude of the effects was likely underestimated because of ceiling effects. The design of the study permitted the animals only 20 training sessions to learn Land-0 and 25 sessions to learn Land-1, thereby providing a ceiling on the maximal number of possible incorrect responses. Further, dogs that did not learn Land-0 within the first 10 sessions were given an additional 10 remedial training days before restarting training. To summarize, these results indicate that the test compound has a robust effect in improving both complex object discrimination learning (Land-0) and allocentric spatial learning (Land-1).

At the completion of the landmark phase of the study, the dogs were tested on a variable delay DNMP task, which provides a measure of visuospatial working memory capacity. Before the subjects were entered in this study, they had been trained on the DNMP task. The animals in the animal treatment group performed slightly better than the control group, but the results did not approach statistical significance; nor did the groups differ statistically when the analysis took into consideration the different baseline performance. Collectively, these data indicate that the cognitive enhancing effectiveness of short-term maintenance on LA+ALCAR varies as a function of task, and possibly prior training on the task.

The absence of an effect on the DNMP task suggests that short-term treatment does not have equivalent effects on all aspects of cognition. Rather, the results point to LA+ALCAR having greater effectiveness in facilitating complex learning than it does in improving short-term memory.

The far superior performance of the dogs given the combination of LA+ALCAR provides compelling evidence of its therapeutic effectiveness in reversing age-associated deficits in learning two different, but interrelated tasks.

This is the first study with dogs to demonstrate positive effects of the test compound on cognition. Two possible mechanisms can account for beneficial effects on learning of short-term maintenance on LA+ALCAR. First, the mechanisms underlying acquisition of the Land-0 and Land-1 tasks are different from those underlying performance on the DNMP and almost certainly also involve different cerebral circuitry. Discrimination learning must depend on the establishment of plastic changes in transmission, probably at synapses—and these changes in turn depend on active cellular processes. New synapse production also involves increased neurotransmitter production, which requires a significant amount of available energy. Virtually all this energy is produced in the mitochondria. This requires structurally and functionally healthy robust mitochondria. In the present study, the animals had been trained and practiced on the DNMP task prior to the start of the study. Thus, in this study, the DNMP task did not assess learning. Since the animals were highly practiced on the task, improved metabolic function would likely have a much-reduced effect on task performance.

A second possible mechanism for the improvement in learning may result from an increased synthesis of the neurotransmitter acetylcholine in the experimental group. The acetyl group of ALCAR is transferred to choline in the presence of enzyme choline-acetyl transferase to produce acetylcholine. Increased levels of this neurotransmitter may be instrumental in promoting new synaptic connections and consequently improved learning capacity.

To conclude, the present results demonstrate that aged beagle dogs maintained on a combination of lipoic acid and acetyl-1-carnitine shown significantly improved learning on two separate tasks, when compared to subjects maintained on a methylcellulose control. The effect was robust, and suggests that the test compound is beneficial in compensating for learning decline associated with increased age. By contrast, LA+ALCAR did not affect performance on a previously learned spatial memory task, indicating that the effectiveness as a cognitive modifying treatment varies as a function of task or prior learning. Long-term treatment with LA+ALCAR is likely to have additional cognitive benefits, slowing or delaying the development of cognitive decline.

### Example 2

This example provides an exemplary composition for use in methods described herein.

The exemplary composition contains: chicken meal, rice flour, wheat flour, corn syrup, water, glycerin, molasses, tapioca starch, acetyl-L-carnitine, gelatin, natural flavor, chicken fat (preserved with mixed tocopherols and citric acid), salt, alpha lipoic acid, phosphoric acid, L-ascorbyl-2-polyphosphate (source of vitamin C), potassium sorbate (a preservative), mono and diglycerides, vitamin E supplement, minerals (zinc proteinate, iron proteinate, copper proteinate, manganese proteinate, cobalt proteinate), calcium propionate (a preservative), garlic powder, and biotin supplement.

The weight percentage of each component in the composition is provided in Table 2. The composition can be made in the forms of dog treats (each treat is about 5 grams).

TABLE 2

| Component | Weight % |
|---|---|
| Chicken meal | 22.00 |
| Rice flour | 15.40 |
| Wheat Flour | 13.00 |
| Corn syrup | 9.00 |
| Glycerin | 6.00 |
| Molasses, dried | 5.00 |
| Tapioca flour | 3.00 |
| Acetyl-L-carnitine | 2.80 |
| Gelatin | 2.50 |
| Chicken liver digest (ddpe) | 2.00 |
| Chicken fat | 2.00 |
| Salt | 1.50 |
| Alpha lipoic acid | 1.15 |
| Phosphoric acid | 1.00 |
| Stay-C vitamin C | 0.40 |
| Potassium sorbate | 0.30 |
| Dimodan | 0.20 |
| NatureOx | 0.15 |
| Vitamin E supplement | 0.10 |
| Chelated mineral premix | 0.10 |
| Mycocurb | 0.10 |
| Garlic powder | 0.10 |
| Biotin supplement | 0.025 |
| Water to balance | 12.17 |

The guaranteed analysis for the composition described herein is provided in Table 3.

TABLE 3

| Component | Amount | |
|---|---|---|
| Crude protein (min) | 22% | |
| Crude fat (min) | 5% | |
| Crude fiber (max) | 1% | |
| Moisture (max) | 26% | |
| Vitamin E | 5.00 | IU/kg |
| Biotin | 1.25 | mg/kg |
| Ascorbic acid | 20.00 | mg/kg |

What is claimed is:

1. A method of improving learning ability of an aged companion animal, comprising administering to the aged companion animal an effective amount of a carnitine and an effective amount of a lipoic acid, wherein the amount of carnitine is about 8-40 mg/kg body weight/day and wherein the amount of lipoic acid is about 4-20 mg/kg body weight/day.

2. The method of claim 1, wherein the amount of carnitine is about 15-30 mg/kg body weight/day and wherein the lipoic acid is about 10-15 mg/kg body weight/day.

3. The method of claim 1, wherein the carnitine and the lipoic acid are administered at least twice daily.

4. The method of claim 1, wherein the carnitine and the lipoic acid are administered at least 4 hours after a meal.

5. The method of claim 1, wherein the improvement of learning ability of the aged companion animal can be observed within less than about one month after the initiation of the administration.

6. The method of claim 5, wherein the improvement of learning ability of the aged companion animal can be observed within less than about five days after the initiation of the administration.

7. The method of claim 1, wherein the carnitine and the lipoic acid are administered orally to the aged companion animal.

8. The method of claim 1, wherein the aged companion animal is an aged dog.

9. The method of claim 8, wherein the aged dog is at least about 7 years old.

10. The method of claim 1, wherein the carnitine is alkanoyl-L-carnitine.

11. The method of claim 10, wherein the carnitine is acetyl-L-carnitine.

12. The method of claim 1, wherein the lipoic acid is a mixture of R-lipoic acid and S-lipoic acid having an R-enatiomer excess of at least about 40%.

13. The method of claim 1, wherein the lipoic acid is R-lipoic acid.

14. The method of claim 1, further comprising administering biotin to the aged companion animal.

15. The method of claim 1, wherein the carnitine and the lipoic acid are administered in a single composition.

16. The method of claim 15, wherein the weight percentage of the carnitine in the composition is at least about 2% and the weight percentage of the lipoic acid in the composition is at least about 1%.

17. The method of claim 15, wherein the composition further comprises biotin.

18. An animal treat for improving learning ability of an aged companion animal comprising a carnitine and a lipoic acid, wherein the amount of carnitine per serving is about 100-500 mg, wherein the amount of lipoic acid per serving is about 50-250 mg, and wherein the animal treat further comprises any one or more of meat by-product, poultry by-product, meat meal, bone meal, chicken or poultry liver digest, chicken or poultry viscera digest, brewer rice, and brewer dry yeast.

19. The animal treat of claim 18, wherein the amount of carnitine per serving is about 200-300 mg.

20. The animal treat of claim 18, wherein the amount of lipoic acid per serving is about 100-150 mg.
